(19) United States
(12) Patent Application Publication
(10) Pub. No.: US 2026/0248435 A1
(43) Pub. Date: Aug. 27, 2026

(54) ARRHYTHMIA TYPE DETERMINATION DEVICE, ARRHYTHMIA TYPE DETERMINATION METHOD, AND RECORDING MEDIUM

(71) Applicants: KANADEVIA CORPORATION, Osaka (JP); NATIONAL CEREBRAL AND CARDIOVASCULAR CENTER, Osaka (JP)

(72) Inventors: Jun YOSHIMATSU, Osaka (JP); Aiko KAKIGANO, Osaka (JP); Hiroki MATSUZAKI, Osaka (JP); Ryo ITOH, Osaka (JP); Masahiro TOMARU, Osaka (JP)

(21) Appl. No.: 19/551,853

(22) Filed: Feb. 27, 2026

(30) Foreign Application Priority Data
Feb. 27, 2025 (JP) 2025-030306

(51) Int. Cl. A61B 5/318; A61B 5/00; A61B 8/02

(52) U.S. Cl. CPC A61B 5/318; A61B 5/726; A61B 8/02

(57) ABSTRACT

Provided is an arrhythmia type determination device including: an obtaining section that obtains a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and a determination section that determines the type of a premature contraction suffered by the target subject, based on the target signal waveform in a period of interest in which the premature contraction is occurring in the heart of the target subject. This allows the arrhythmia type determination device to accurately determine the type of premature contraction from an image obtained by image-capturing of a heart.

This Nonprovisional application claims priority under 35 U.S.C. § 119 on Patent Application No. 2025-030306 filed in Japan on Feb. 27, 2025, the entire contents of which are hereby incorporated by reference.

### TECHNICAL FIELD

The present invention relates to a technique for determining the type of arrhythmia.

### BACKGROUND ART

There is a widely-used technology for grasping a relation between the left and right sides of the heart and a relation between atria and ventricles from a moving image of a heart obtained by ultrasonography. For example, Patent Literature 1 discloses an ultrasound diagnostic device that identifies a group of boundary positions between a plurality of heart chambers in a moving image obtained by ultrasonography and obtains, on the basis of a result of tracking the group of the boundary positions, the boundary positions between the plurality of heart chambers over a period of at least one heartbeat.

CITATION LIST

### Patent Literature

### Patent Literature 1

Japanese Patent Application Publication Tokukai No. 2022-149097

### SUMMARY OF INVENTION

### Technical Problem

Conventionally, electrocardiograms are mainly used for diagnosing arrhythmia. However, it may be difficult to determine the type of premature contraction that has occurred, and there has been room for improvement in terms of determination accuracy. It should be noted that the premature contraction is the type of arrhythmia, in which an abnormal stimulation occurs in the heart, and a pulsation due to an additional contraction is added besides a regular pulsation.

An object of an aspect of the present invention is to accurately determine the type of the premature contraction from an image obtained by image-capturing of the heart.

### Solution to Problem

An arrhythmia type determination device according to an aspect of the present invention includes: an obtaining section that obtains a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and a determination section that determines the type of a premature contraction suffered by the target subject, based on the target signal waveform in a period of interest in which the premature contraction is occurring in the heart of the target subject.

An arrhythmia type determination method according to an aspect of the present invention is executed by one or more information processing devices, the method including: an obtaining step of obtaining a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and a determination step of determining the type of a premature contraction suffered by the target subject, based on the target signal waveform in a period of interest in which the premature contraction is occurring in the heart of the target subject.

The arrhythmia type determination device in accordance with the foregoing aspects of the present invention may be achieved by a computer. In such a case, the present invention encompasses: a control program for the arrhythmia type determination device that causes a computer to operate as each of the sections (software elements) of the arrhythmia type determination device so that the arrhythmia type determination device can be achieved by the computer; and a computer-readable storage medium storing the control program therein.

ADVANTAGEOUS EFFECTS OF INVENTION

With an aspect of the present invention, it is possible to accurately determine the type of premature contraction from an image obtained by image-capturing of a heart.

### BRIEF DESCRIPTION OF DRAWINGS

FIG. 1 is a diagram showing a configuration example of an arrhythmia type determination system according to an embodiment of the present invention.

FIG. 2 is a view showing an example in which respective regions of a left atrium, a left ventricle, a right atrium, and a right ventricle are detected from an ultrasound image of a heart.

FIG. 3 is a diagram showing an example in which respective regions of a left atrium, a left ventricle, a right atrium, and a right ventricle are detected from an ultrasound image of a heart of a target subject not suffering from arrhythmia.

FIG. 4 is a diagram showing an example of a target signal waveform including a period in which a premature ventricular contraction (PVC) is occurring.

FIG. 5 is a diagram showing an example of a target signal waveform including a period in which a premature atrial contraction (PAC) is occurring.

FIG. 6 is a block diagram showing an example of the configuration of the main parts of an arrhythmia type determination device according to Embodiment 1 of the present invention.

FIG. 7 is a diagram for explaining an example of processing for detecting an increasing period and a decreasing period from a set of an atrial waveform and a ventricular waveform.

FIG. 8 is a flowchart showing an example of a flow of processing performed by the arrhythmia type determination device.

FIG. 9 is a block diagram showing an example of the configuration of the main parts of an arrhythmia type determination device according to Embodiment 2 of the present invention.

FIG. 10 is a diagram showing an example in which the shape of a single-beat atrial waveform and the shape of a single-beat ventricular waveform are compared.

FIG. 11 is a diagram showing another example in which the shape of a single-beat atrial waveform and the shape of a single-beat ventricular waveform are compared.

FIG. 12 is a flowchart showing an example of a flow of processing performed by the arrhythmia type determination device.

FIG. 13 is a block diagram showing an example of the configuration of the main parts of an arrhythmia type determination device according to Embodiment 3 of the present invention.

FIG. 14 is a diagram showing a state in which a deletion of a ventricular contraction corresponding to an atrial contraction is detected from a set of an atrial waveform and a ventricular waveform.

FIG. 15 is a flowchart showing an example of a flow of processing performed by the arrhythmia type determination device.

FIG. 16 is a block diagram showing an example of the configuration of the main parts of an arrhythmia type determination device according to Embodiment 4 of the present invention.

### DESCRIPTION OF EMBODIMENTS

### Embodiment 1

The following description will discuss an embodiment of the present invention in detail.

### Overview of Arrhythmia Type Determination Device 1

An arrhythmia type determination device 1 according to an embodiment of the present invention determines the type of a premature contraction suffered by a target subject, based on a target signal waveform in a period of interest in which the premature contraction is occurring in a heart of the target subject. It should be noted here that the target signal waveform is generated by analyzing an image of the heart of the target subject, and includes at least one of the following sets (1) and (2).
(1) A set of a signal waveform indicating a time-series change in the area of the region of a left atrium (left atrial waveform) and a signal waveform indicating a time-series change in the area of the region of a left ventricle (left ventricular waveform).
(2) A set of a signal waveform indicating a time-series change in the area of the region of a right atrium (right atrial waveform) and a signal waveform indicating a time-series change in the area of the region of a right ventricle (right ventricular waveform).

In the following description, the “atrial waveform” is intended to mean a signal waveform indicating a time-series change in the area of the region of an atrium without particularly limiting whether the atrium is the left atrium or the right atrium. In addition, in the following description, the “ventricular waveform” is intended to mean a signal waveform indicating a time-series change in the area of the region of a ventricle without particularly limiting whether the ventricle is the left ventricle or the right ventricle.

The premature contraction is an arrhythmia in which the heart contracts early out of an original cycle due to an abnormal stimulation generated in the heart, and refers to a state in which an early pulse is occasionally mixed with a normal and regular pulse. Main types of the premature contraction include premature ventricular contraction (PVC) and premature atrial contraction (PAC). PVC is a premature contraction in which a ventricle is activated before a normal pulsation occurs due to an abnormal electrical stimulation generated in the ventricle, whereby a contraction deviating from a normal contraction rhythm occurs. On the other hand, PAC is a premature contraction in which an atrium is activated before a normal pulsation occurs due to an electrical stimulation generated at an abnormal site, whereby a contraction deviating from a normal contraction rhythm occurs.

The inventors have found that the type of the premature contraction occurring in the heart can be accurately determined based on a signal waveform indicating a time-series change in the area of a region, which is at least one of the left atrium, the left ventricle, the right atrium, and the right ventricle included in an image obtained by image-capturing of the heart. The arrhythmia type determination device 1 can accurately determine the type of the premature contraction occurring in the heart of the target subject from the image obtained by image-capturing of the heart of the target subject.

The target subject may be an animal having a heart and a fetus thereof. For example, the target subject may be a human fetus. In this case, the area of each atrium and each ventricle may be calculated based on an ultrasound image including images of a plurality of frames obtained by image-capturing of the fetus. The heart of the fetus is captured in the ultrasound image obtained by image-capturing of the fetus, and the area of each region of the heart of the fetus can be calculated from the ultrasound image. Processing for calculating the area of each region of the heart from the ultrasound image will be described later with a specific example.

### Configuration of Arrhythmia Type Determination System 100

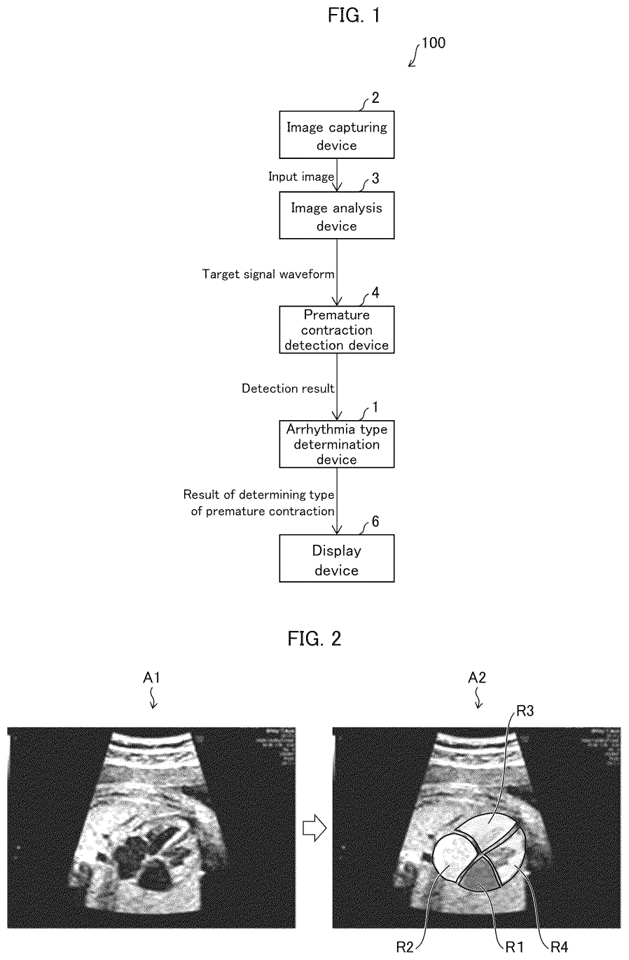

First, a configuration of an arrhythmia type determination system 100 including the arrhythmia type determination device 1 according to an embodiment of the present invention will be described with reference to FIG. 1. FIG. 1 is a diagram showing a configuration example of the arrhythmia type determination system 100. The arrhythmia type determination system 100 may include an image capturing device 2, an image analysis device 3, a premature contraction detection device 4, the arrhythmia type determination device 1, and a display device 6.

### Image Capturing Device 2

The image capturing device 2 may be an ultrasound image capturing device capable of non-invasively capturing an image of the inside of the body of the target subject. That is, an input image captured by the image capturing device 2 may be, for example, an ultrasound image such as an echo image for tomographic image formation. The image capturing device 2 may be communicably connected to the image analysis device 3 as illustrated, and in this case, the image analysis device 3 may directly obtain the input image from the image capturing device 2. It should be noted that the input image may be stored in an image management device (not shown) in association with target subject information (for example, electronic medical record information) for each target subject, and in this case, the arrhythmia type determination device 1 may obtain the input image from the image management device.

### Image Analysis Device 3

The image analysis device 3 detects respective regions of the left atrium, the left ventricle, the right atrium, and the right ventricle of the heart of the target subject captured in the input image. Furthermore, the image analysis device 3 calculates the area of each detected region and generates a target signal waveform indicating a time-series change in the area of each region.

The image analysis device 3 may be installed in a facility (for example, a medical facility) where the image capturing device 2 is installed, or may be installed in a remote place. When the image analysis device 3 is installed in a remote place, the image analysis device 3 may obtain the input image by communication via a communication network such as the Internet.

Processing for detecting each region corresponding to each atrium and each ventricle of the heart from the input image will be described with reference to FIG. 2. FIG. 2 is a view showing an example in which respective regions of a left atrium, a left ventricle, a right atrium, and a right ventricle are detected from an ultrasound image of a heart. An image A1 shown in FIG. 2 is an ultrasound image of the heart, and an image A2 shows detection results of respective regions R1 to R4 superimposed on the ultrasound image.

The heart is captured in a region slightly below the center in the image A1, and an outer shape thereof is visible, and it is also visible that the inside of the heart is divided into a plurality of sections. As described above, in the ultrasound image, each of two ventricles and each of two atria included in the heart can be visually recognized as closed regions. Therefore, by analyzing the ultrasound image of the heart, it is possible to detect the respective regions R1 to R4 of the left atrium, the left ventricle, the right atrium, and the right ventricle.

For example, machine learning can be performed using training data in which labels such as left atrium, left ventricle, right atrium, and right ventricle are attached as ground truth data to regions corresponding to the left atrium, the left ventricle, the right atrium, and the right ventricle of the heart captured in the ultrasound image of the heart. The label can also be referred to as an annotation. By such machine learning, a trained model capable of detecting the respective regions R1 to R4 of the left atrium, the left ventricle, the right atrium, and the right ventricle from the ultrasound image of the heart can be constructed. For example, by constructing a trained model of a convolutional neural network and using the trained model, highly accurate region detection becomes possible.

The image A2 shown in FIG. 2 shows a result of detection using such a trained model. The detected regions R1 to R4 are regions detected as the right ventricle, the left ventricle, the left atrium, and the right atrium, respectively. By detecting the respective regions R1 to R4, it becomes possible to calculate the area of each of the regions R1 to R4.

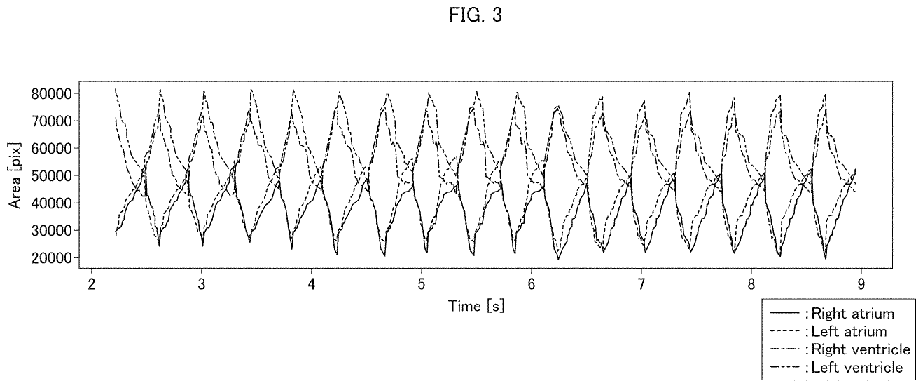

For example, the area of each of the regions R1 to R4 can be represented by the number of pixels included in each of the detected regions R1 to R4. By calculating the area of each of the regions R1 to R4 of the left atrium, the left ventricle, the right atrium, and the right ventricle of the heart of the target subject in each frame of the ultrasound image, the target signal waveform indicating the time-series change in the area of each of the regions R1 to R4 can be obtained. FIG. 3 is a diagram showing an example in which respective regions of the left atrium, the left ventricle, the right atrium, and the right ventricle are detected from the ultrasound image of the heart of the target subject not suffering from arrhythmia. In the case of a heart not suffering from arrhythmia, as shown in FIG. 3, the area of each atrium and each ventricle repeatedly increases and decreases at a substantially constant cycle. Also, a heartbeat rhythm is reproduced in which the area of the ventricle decreases during a period in which the area of the region of the atrium increases, and the area of the ventricle increases during a period in which the area of the region of the atrium decreases.

### Premature Contraction Detection Device 4

The premature contraction detection device 4 detects a period of interest in which a premature contraction such as PVC or PAC is occurring in the heart of the target subject, using the target signal waveform obtained from the image analysis device 3. It should be noted here that the length of the period of interest is desirably longer than a period corresponding to a predetermined number of times (for example, three or more times) of pulsations. As an example, the period of interest may be 3 seconds (corresponding to 8 to 10 pulsations), 5 seconds (corresponding to 12 to 15 pulsations), or 10 seconds (corresponding to 24 to 30 pulsations).

For example, the premature contraction detection device 4 may detect the period of interest by executing the following step (a) and step (b).
Step (a): Comparing a time interval from a time point when the area indicated by the target signal waveform becomes a reference value set based on a displacement of the target signal waveform until the area indicated by the target signal waveform next becomes the reference value, with a predetermined threshold for determining presence or absence of the premature contraction. It should be noted here that the reference value may be any value within a range in which the target signal waveform is displaced, and may be, for example, a center value of the displacement in the target signal waveform.
Step (b): Detecting the period of interest based on a comparison result obtained in the step (a).

When the period of interest is detected in the target signal waveform obtained from the image analysis device 3, the premature contraction detection device 4 may output period-of-interest information indicating a position of the period of interest in the target signal waveform, together with the target signal waveform, to the arrhythmia type determination device 1 described later. In this case, the arrhythmia type determination device 1 determines the type of the premature contraction suffered by the target subject, based on a waveform in the period of interest specified by the period-of-interest information among the target signal waveforms obtained from the premature contraction detection device 4.

Alternatively, when the period of interest is detected in the target signal waveform obtained from the image analysis device 3, the premature contraction detection device 4 may cut out a partial waveform corresponding to the period of interest (that is, the target signal waveform in the period of interest) from the target signal waveform, and output the partial waveform to the arrhythmia type determination device 1 described later. The cut-out partial waveform includes one or more periods including a waveform serving as a basis for determining that the premature contraction is occurring. In this case, the arrhythmia type determination device 1 determines the type of the premature contraction suffered by the target subject based on the partial waveform obtained from the premature contraction detection device 4.

It should be noted that the premature contraction detection device 4 is not limited to the configuration shown in FIG. 1. For example, the premature contraction detection device 4 may be configured to obtain, in addition to the target signal waveform obtained from the image analysis device 3, at least one of pulse data obtained by measuring a pulse of the target subject during capturing of the input image used for generating the target signal waveform, and electrocardiogram data recording a pulsation of the heart of the target subject. In this case, the premature contraction detection device 4 may detect the period of interest by applying a known detection criterion based on at least one of pulse wave data and an electrocardiogram. For example, when the period of interest is detected from the electrocardiogram of the target subject, the premature contraction detection device 4 may cut out the target signal waveform of the target subject in the detected period of interest from the target signal waveform, and output the target signal waveform to the arrhythmia type determination device 1 described later.

It should be noted that the premature contraction detection device 4 may be communicably connected to the image analysis device 3 as illustrated, and in this case, the premature contraction detection device 4 may directly obtain the target signal waveform from the image analysis device 3. It should be noted that the target signal waveform generated by the image analysis device 3 may be stored in, for example, a portable recording medium, and in this case, the premature contraction detection device 4 may read the target signal waveform from the recording medium. Alternatively, the target signal waveform generated by the image analysis device 3 may be stored in, for example, any storage device (not shown) in association with the target subject information for each target subject, and in this case, the premature contraction detection device 4 may obtain the target signal waveform from the storage device.

The premature contraction detection device 4 may be installed in a facility (for example, a medical facility) where the image analysis device 3 is installed, or may be installed in a remote location. When installed in a remote location, the premature contraction detection device 4 may obtain the target signal waveform generated by the image analysis device 3 by communication via a communication network such as the Internet.

A part of processing performed by the premature contraction detection device 4 may be executed by another computer. That is, the processing performed by the premature contraction detection device 4 may be executed by one or more information processing devices. Alternatively, the premature contraction detection device 4 may have a function of the image analysis device 3. For example, when the premature contraction detection device 4 also has the function of the image analysis device 3, the image analysis device 3 is omitted from components of the arrhythmia type determination system 100.

The following description will discuss, as an example, a configuration in which the arrhythmia type determination device 1 obtains the partial waveform (that is, the target signal waveform in the period of interest) cut out by the premature contraction detection device 4, and determines the type of the premature contraction occurring in the heart of the target subject based on the target signal waveform. In the following description, when described as “target signal waveform”, the target signal waveform in the period of interest is intended, and includes a set of an atrial waveform and a ventricular waveform, for example, at least one of the following sets (1) and (2).
(1) A set of a left atrial waveform and a left ventricular waveform.
(2) A set of a right atrial waveform and a right ventricular waveform.

### Arrhythmia Type Determination Device 1

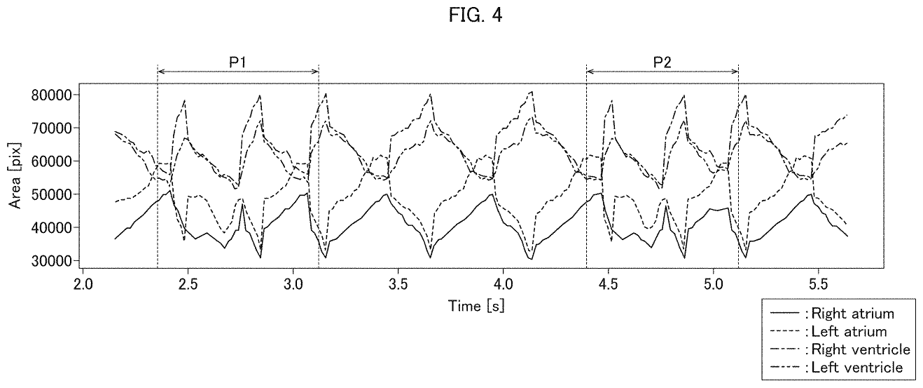

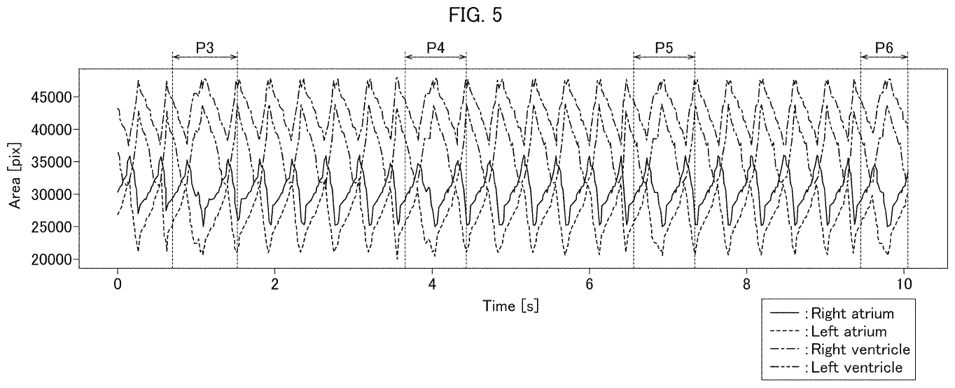

FIG. 4 is a diagram showing an example of the target signal waveform including a period in which premature ventricular contraction (PVC) is occurring, and PVC is occurring in periods P1 and P2. Meanwhile, FIG. 5 is a diagram showing an example of the target signal waveform including a period in which premature atrial contraction (PAC) is occurring, and PAC is occurring in periods P3 to P6. It should be noted here that PVC is a premature contraction in which a ventricle is activated before a normal pulsation occurs due to an abnormal electrical stimulation generated in the ventricle, whereby a contraction deviating from a normal contraction rhythm occurs. On the other hand, PAC is a premature contraction in which an atrium is activated before a normal pulsation occurs due to an electrical stimulation generated at an abnormal site, whereby a contraction deviating from a normal contraction rhythm occurs.

The inventors have found that a clear difference is recognized in the target signal waveform between a case where PVC occurs in the heart of the target subject and a case where PAC occurs in the heart of the target subject.

For example, in the target signal waveform shown in FIG. 3, the area indicated by the ventricular waveform always decreases during a period in which the area indicated by the atrial waveform increases, and the area indicated by the ventricular waveform always increases during a period in which the area indicated by the atrial waveform decreases. Meanwhile, in the target signal waveform shown in FIG. 4 (particularly in the periods P1 and P2), there exist a period in which both the area indicated by the atrial waveform and the area indicated by the ventricular waveform increase (hereinafter referred to as “increasing period”), and a period in which both the area indicated by the atrial waveform and the area indicated by the ventricular waveform decrease (hereinafter referred to as “decreasing period”). On the other hand, the increasing period and the decreasing period do not exist in the target signal waveform shown in FIG. 5.

In addition, in the target signal waveform shown in FIG. 3, a similarity between the shape of a single-beat atrial waveform corresponding to a single heartbeat of the heart in the atrial waveform and the shape of a single-beat ventricular waveform corresponding to the single heartbeat in the ventricular waveform is always high, and the similarity does not drop below a predetermined reference value. Meanwhile, in the target signal waveform shown in FIG. 4 (particularly in the periods P1 and P2), the similarity between the shape of the single-beat atrial waveform and the shape of the single-beat ventricular waveform often falls below the predetermined reference value. On the other hand, in the target signal waveform shown in FIG. 5, even in the periods P3 to P6, the similarity between the shape of the single-beat atrial waveform and the shape of the single-beat ventricular waveform rarely drops below the predetermined reference value.

In addition, in the target signal waveform shown in FIG. 3, if the atrium contracts, the ventricle always contracts subsequently, and if the ventricle contracts, the atrium always contracts subsequently. Meanwhile, in the target signal waveform shown in FIG. 4 (particularly in the periods P1 and P2), a deletion of a ventricular contraction corresponding to an atrial contraction and a deletion of an atrial contraction corresponding to a ventricular contraction are observed. On the other hand, in the target signal waveform shown in FIG. 5, even in the periods P3 to P6, the deletion of the ventricular contraction corresponding to the atrial contraction and the deletion of the atrial contraction corresponding to the ventricular contraction are not observed.

Therefore, the inventors have discovered that the type of the premature contraction occurring in the heart of the target subject can be accurately determined based on the target signal waveform. The arrhythmia type determination device 1, using the target signal waveform, determines the type of the premature contraction suffered by the target subject. With the arrhythmia type determination device 1, it is possible to output a more accurate determination result as compared with a case where the type of the premature contraction is determined using an electrocardiogram.

The arrhythmia type determination device 1 may be communicably connected to the premature contraction detection device 4 as illustrated, and in this case, the arrhythmia type determination device 1 may directly obtain the target signal waveform from the premature contraction detection device 4. Alternatively, the arrhythmia type determination device 1 may be communicably connected to the image analysis device 3 and the premature contraction detection device 4, and in this case, the arrhythmia type determination device 1 may obtain the target signal waveform from the image analysis device 3 and obtain the period-of-interest information from the premature contraction detection device 4.

The target signal waveform may be stored in, for example, a portable recording medium, and in this case, the arrhythmia type determination device 1 may read the target signal waveform from the recording medium. Alternatively, the target signal waveform may be stored in, for example, any storage device (not shown) in association with the target subject information for each target subject, and in this case, the arrhythmia type determination device 1 may obtain the target signal waveform from the storage device.

The arrhythmia type determination device 1 may be installed in a facility (for example, a medical facility) where the premature contraction detection device 4 is installed, or may be installed in a remote location. When installed in a remote location, the arrhythmia type determination device 1 may obtain the target signal waveform from the premature contraction detection device 4 by communication via a communication network such as the Internet.

A part of processing performed by the arrhythmia type determination device 1 may be executed by another computer. That is, the processing performed by the arrhythmia type determination device 1 may be executed by one or more information processing devices.

The arrhythmia type determination device 1 may have a function of the premature contraction detection device 4. For example, when the arrhythmia type determination device 1 also has the function of the premature contraction detection device 4, the premature contraction detection device 4 is omitted from components of the arrhythmia type determination system 100. Alternatively, the arrhythmia type determination device 1 may have a function of the image analysis device 3 and the function of the premature contraction detection device 4. For example, when the arrhythmia type determination device 1 also has the function of the image analysis device 3 and the function of the premature contraction detection device 4, the image analysis device 3 and the premature contraction detection device 4 are omitted from components of the arrhythmia type determination system 100.

### Display Device 6

The display device 6 is a device capable of displaying various types of information output from the arrhythmia type determination device 1. The display device 6 may display the input image output from the image capturing device 2, the target signal waveform output from the premature contraction detection device 4, and the like, in addition to the various types of information output from the arrhythmia type determination device 1.

### Configuration of Arrhythmia Type Determination Device 1

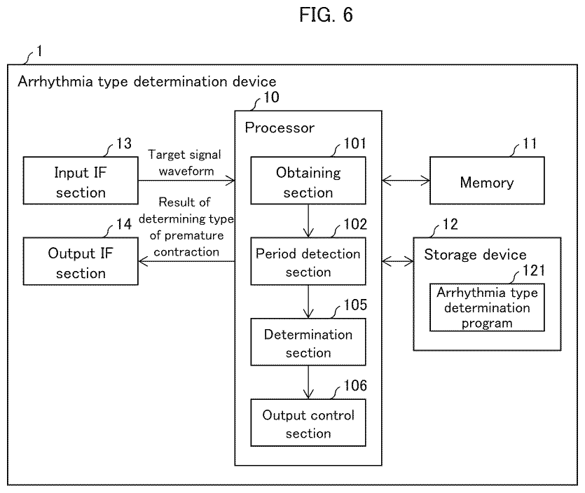

Subsequently, a configuration of the arrhythmia type determination device 1 according to an embodiment of the present invention will be described with reference to FIG. 6. FIG. 6 is a block diagram showing an example of the configuration of the main parts of the arrhythmia type determination device 1. The following description will discuss, as an example, the arrhythmia type determination device 1 having a function of determining the type of the premature contraction suffered by the target subject from the target signal waveform obtained from the premature contraction detection device 4.

As illustrated, the arrhythmia type determination device 1 is, for example, a computer including a processor 10, a memory 11, and a storage device 12. The arrhythmia type determination device 1 may be a personal computer, a server, or a workstation. The processor 10 functions as each section from an obtaining section 101 to an output control section 106 described later by loading an arrhythmia type determination program 121 stored in the storage device 12 into the memory 11 and executing the program.

The processor 10 can be achieved by a logic circuit (hardware) formed in an integrated circuit (IC chip) or the like, or can be achieved by software. When achieved by software, the processor 10 may be configured by, for example, a CPU (Central Processing Unit), may be configured by a GPU (Graphics Processing Unit), or may be configured by a combination thereof. In this case, the software is stored in the storage device 12. Then, the processor 10 reads the software into the memory 11 and executes the software.

Both the memory 11 and the storage device 12 are storage devices that store various types of data used by the arrhythmia type determination device 1. The memory 11 is a storage device capable of writing and reading data at a higher speed than the storage device 12. The storage device 12 has a larger data storage capacity than the memory 11. As the memory 11, for example, a high-speed access memory such as an SDRAM (Synchronous Dynamic Random-Access Memory) can be applied. As the storage device 12, for example, an HDD (Hard Disk Drive), an SSD (Solid-State Drive), an SD (Secure Digital) card, an eMMC (embedded Multi-Media Controller), or the like can be applied.

In addition, the arrhythmia type determination device 1 includes an input IF section 13 and an output IF section 14 as an interface (IF) with an external device. The input IF section 13 is an interface for accepting an input signal from an input device such as a keyboard or a mouse, or obtaining various types of information and data from an external device. The input IF section 13 is, for example, an interface for connecting the premature contraction detection device 4 to the input IF section 13 and obtaining the target signal waveform or the like from the premature contraction detection device 4. The output IF section 14 is an interface for outputting a determination result of the type of the premature contraction or the like to an external device. The output IF section 14 can also connect a display device to the output IF section 14, for example, and cause the display device to display the determination result of the type of the premature contraction or the like.

The processor 10 functions as each of the obtaining section 101 that obtains the target signal waveform from the premature contraction detection device 4, a period detection section 102, a determination section 105, and the output control section 106 by executing the arrhythmia type determination program 121.

The period detection section 102 detects an increasing period and a decreasing period in the obtained target signal waveform. More specifically, the period detection section 102 calculates a corrected atrial waveform and a corrected ventricular waveform obtained by removing noise from the atrial waveform and the ventricular waveform, respectively. For noise removal, known filter processing such as a moving average filter, a fast Fourier transform (FFT) filter, or a Gaussian filter can be applied.

Then, the period detection section 102 detects, as the increasing period, a period in which both the area indicated by the corrected atrial waveform and the area indicated by the corrected ventricular waveform increase for a predetermined time or more in the period of interest. In addition, the period detection section 102 detects, as the decreasing period, a period in which both the area indicated by the corrected atrial waveform and the area indicated by the corrected ventricular waveform decrease for a predetermined time or more in the period of interest.

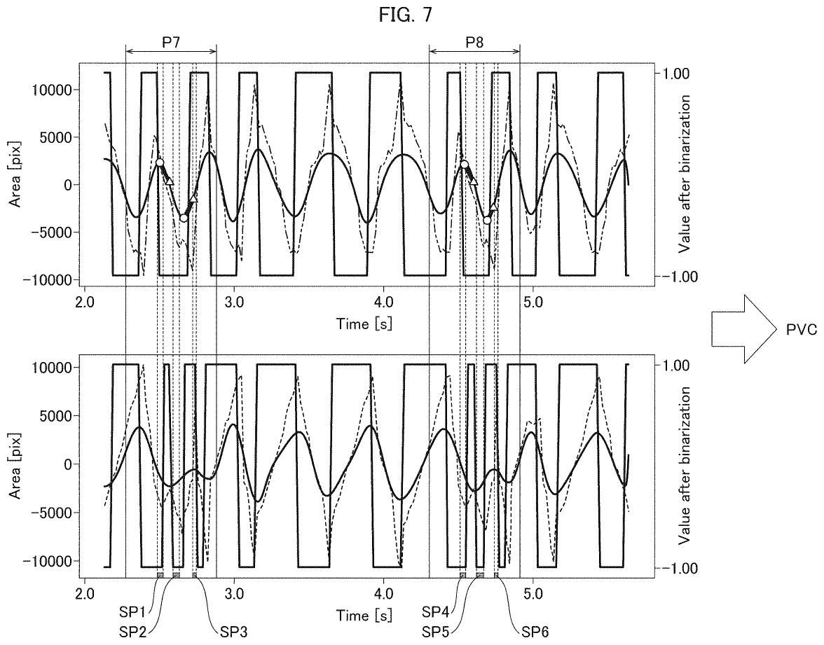

FIG. 7 is a diagram for explaining an example of processing for detecting the increasing period and the decreasing period from the set of the atrial waveform and the ventricular waveform. In FIG. 7, a period P7 and a period P8 are periods in which the premature contraction is occurring. In FIG. 7, a value on a vertical axis indicates a magnitude of an area change at the time of contraction and at the time of expansion of each region of the atrium and the ventricle.

In the upper part of FIG. 7, a two-dot chain line is the left ventricular waveform of the target signal waveform, and a solid curve is the corrected ventricular waveform obtained by removing noise from the left ventricular waveform. In addition, in the upper part of FIG. 7, a graph obtained by binarizing an increase/decrease pattern of the area indicated by the corrected ventricular waveform, obtained by giving “1” when the corrected ventricular waveform indicates an increase in area and giving “−1” when the corrected ventricular waveform indicates a decrease in area, is also superimposed. Meanwhile, in the lower part of FIG. 7, a broken line is the left atrial waveform of the target signal waveform, and a solid line is the corrected atrial waveform obtained by removing noise from the left atrial waveform.

In the lower part of FIG. 7, a graph obtained by binarizing an increase/decrease pattern of the area indicated by the left atrial waveform, obtained by giving “1” when the area transitions from a local minimum to a local maximum in the left atrial waveform and giving “−1” when the area transitions from a local maximum to a local minimum, is also superimposed. It should be noted here that among local maximums and local minimums of the area in the left atrial waveform, those occurring in a time shorter than a predetermined time (for example, 0.1 seconds) are ignored.

Based on the corrected ventricular waveform shown in the upper part of FIG. 7 and the corrected atrial waveform shown in the lower part, the period detection section 102 detects, for example, periods indicated by thick lines having white circles and white triangles shown in the upper part as end points, respectively, as the increasing period and the decreasing period.

Alternatively, the period detection section 102 may detect the increasing period and the decreasing period based on rough increase/decrease patterns of the atrial waveform and the ventricular waveform of the target signal waveform. For example, the period detection section 102 may create a graph (lower part of FIG. 7) obtained by binarizing the increase/decrease pattern of the area indicated by the left atrial waveform also for the left ventricular waveform, and detect the increasing period and the decreasing period by comparing these graphs.

The determination section 105 determines the type of the premature contraction suffered by the target subject based on the target signal waveform. Specifically, the determination section 105 determines the type of the premature contraction suffered by the target subject based on a detection status of the increasing period and the decreasing period. That is, the determination section 105 determines that the target subject is suffering from PVC when either the increasing period or the decreasing period is detected in the period of interest, and determines that the target subject is suffering from PAC when neither the increasing period nor the decreasing period is detected in the period of interest. For example, when the increasing period and the decreasing period are detected from the target signal waveform as shown in FIG. 7, the determination section 105 determines that the premature contraction suffered by the target subject is PVC. On the other hand, when neither the increasing period nor the decreasing period is detected from the target signal waveform, the determination section 105 determines that the premature contraction suffered by the target subject is PAC.

Returning to FIG. 6, the output control section 106 causes various output devices to output the determination result by the determination section 105. For example, when the display device 6 is connected via the output IF section 14, the output control section 106 may cause the display device 6 to display the determination result. It should be noted that any mode of outputting the determination result can be employed, and the output control section 106 may output the determination result by display output, audio output, print output, a combination thereof, or the like.

### Processing Performed by Arrhythmia Type Determination Device 1

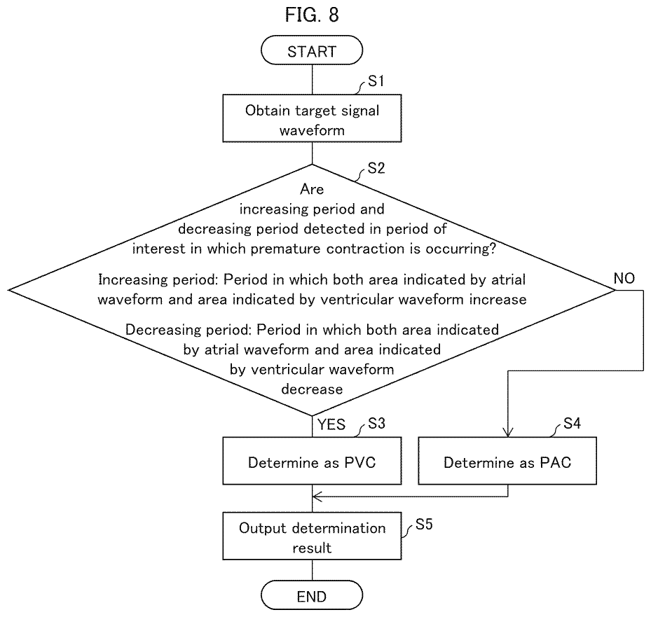

Next, processing (arrhythmia type determination method) performed by the arrhythmia type determination device 1 will be described with reference to FIG. 8. FIG. 8 is a flowchart showing an example of a flow of processing performed by the arrhythmia type determination device 1.

First, the obtaining section 101 obtains the target signal waveform (step S1: obtaining step). Next, the period detection section 102 detects the increasing period and the decreasing period in the period of interest in which the premature contraction is occurring. Then, when the increasing period and the decreasing period are detected (YES in step S2), the determination section 105 determines that the premature contraction suffered by the target subject is PVC (step S3: determination step). On the other hand, when neither the increasing period nor the decreasing period is detected (NO in step S2), the determination section 105 determines that the premature contraction suffered by the target subject is PAC (step S4: determination step). The output control section 106 causes the output device (for example, the display device 6) to output the determination result by the determination section 105 (step S5: output step).

With the above configuration, the arrhythmia type determination device 1 can accurately determine the type of the premature contraction suffered by the target subject from the image obtained by image-capturing of the heart of the target subject. For example, the arrhythmia type determination device 1 can estimate whether the type of the premature contraction of the fetus is “PVC” or “PAC” with a high accuracy rate.

### Embodiment 2

Another embodiment of the present invention will be discussed below. For convenience of description, members having the same functions as the members described in the above embodiment are denoted by the same reference numerals, and description thereof will not be repeated.

The arrhythmia type determination device 1 according to the above embodiment is configured to be capable of determining the type of the premature contraction suffered by the target subject based on whether or not the increasing period in which both the atrial waveform and the ventricular waveform increase and the decreasing period in which both the atrial waveform and the ventricular waveform decrease are detected in the target signal waveform. Meanwhile, an arrhythmia type determination device 1a according to the present embodiment is configured to determine the type of the premature contraction suffered by the target subject based on a similarity between the shapes of one or more atrial waveforms and ventricular waveforms corresponding to respective single heartbeats of the heart of the target subject from the target signal waveform.

Similarly to the above embodiment, the following description will discuss, as an example, the arrhythmia type determination device 1a having a function of determining the type of the premature contraction suffered by the target subject from the target signal waveform obtained from the premature contraction detection device 4. However, the arrhythmia type determination device 1a may also have the function of the image analysis device 3 and the function of the premature contraction detection device 4. In this case, the arrhythmia type determination device 1a can generate the target signal waveform from the input image obtained from the image capturing device 2, and determine the type of the premature contraction suffered by the target subject.

The following description will discuss, as an example, a configuration in which the arrhythmia type determination device 1a obtains the partial waveform (that is, the target signal waveform in the period of interest) cut out by the premature contraction detection device 4, and determines the type of the premature contraction occurring in the heart of the target subject based on the target signal waveform.

### Configuration of Arrhythmia Type Determination Device 1a

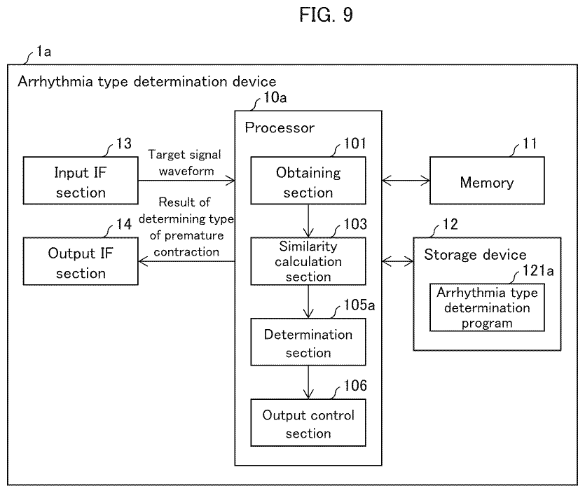

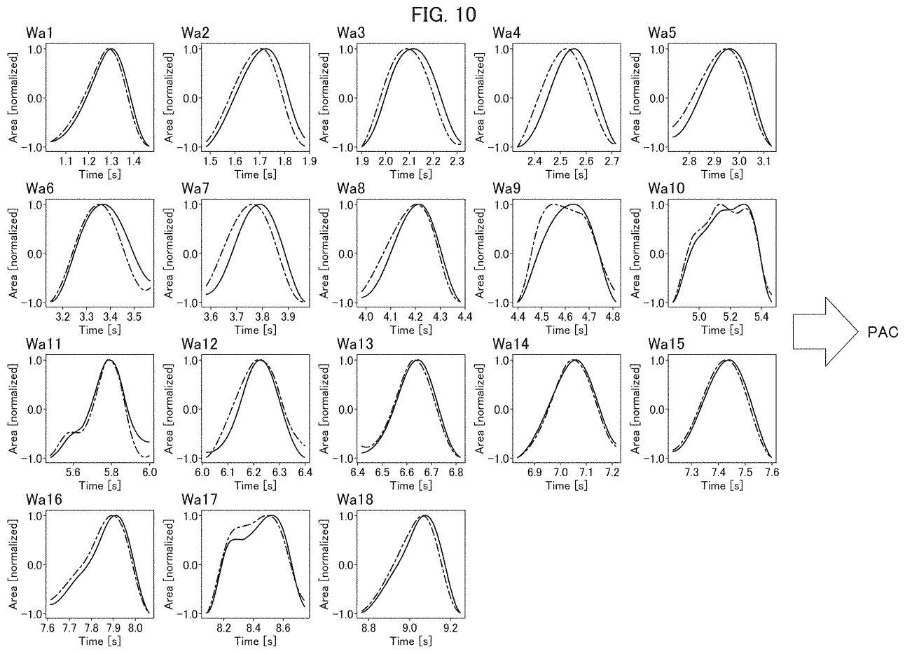

A configuration of an arrhythmia type determination device 1a according to an embodiment of the present invention will be described with reference to FIG. 9. FIG. 9 is a block diagram showing an example of the configuration of the main parts of the arrhythmia type determination device 1a.

As illustrated, the arrhythmia type determination device 1a is, for example, a computer including a processor 10a, a memory 11, and a storage device 12. The arrhythmia type determination device 1a may be a personal computer, a server, or a workstation. The processor 10a functions as each section from an obtaining section 101 to an output control section 106 described later by loading an arrhythmia type determination program 121a stored in the storage device 12 into the memory 11 and executing the arrhythmia type determination program 121a.

The processor 10a can be achieved by a logic circuit (hardware) formed in an integrated circuit (IC chip) or the like, or can be achieved by software. When achieved by software, the processor 10a may be configured by, for example, a CPU, may be configured by a GPU, or may be configured by a combination thereof. In this case, the software is stored in the storage device 12. Then, the processor 10a reads the software into the memory 11 and executes the software.

The processor 10a functions as each of the obtaining section 101 that obtains a target signal waveform from a premature contraction detection device 4, a similarity calculation section 103, a determination section 105a, and the output control section 106 by executing the arrhythmia type determination program 121a.

The similarity calculation section 103 calculates a similarity by comparing the shape of an atrial waveform and the shape of a ventricular waveform in a period of interest.

More specifically, the similarity calculation section 103 first calculates a corrected atrial waveform and a corrected ventricular waveform obtained by removing noise from the atrial waveform and the ventricular waveform, respectively. For noise removal, known filter processing such as a moving average filter, an FFT filter, or a Gaussian filter can be applied.

Subsequently, the similarity calculation section 103 obtains a plurality of single-beat atrial waveforms corresponding to respective single heartbeats of a heart of a target subject from the corrected atrial waveform in the period of interest. In addition, the similarity calculation section obtains a plurality of single-beat ventricular waveforms corresponding to respective single heartbeats of the heart of the target subject from the corrected ventricular waveform in the period of interest.

In order to obtain the single-beat atrial waveform, the similarity calculation section 103 may detect time points at which an atrium turns from a systole to a diastole (local minimum points in the atrial waveform) from the corrected atrial waveform, and divide the atrial waveform at these time points. Meanwhile, in order to obtain the single-beat ventricular waveform, the similarity calculation section 103 may detect time points at which a ventricle turns from the systole to the diastole (local minimum points in the ventricular waveform) from the corrected ventricular waveform, and divide the ventricular waveform at these time points.

The similarity calculation section 103 may be configured to obtain the single-beat atrial waveform and the single-beat ventricular waveform by detecting given time points serving as delimiters of respective pulsations of the heart from the corrected atrial waveform and the corrected ventricular waveform and dividing the atrial waveform and the ventricular waveform. For example, when obtaining the single-beat atrial waveform from the corrected atrial waveform, the similarity calculation section 103 may be configured to detect a time point at which the area of the region of the atrium becomes a predetermined value in the diastole in the corrected atrial waveform. In this case, the similarity calculation section 103 obtains, as the single-beat atrial waveform, a waveform from the time point at which the area of the region of the atrium becomes the predetermined value in the diastole to a time point at which the area of the region of the atrium becomes the predetermined value in the next diastole. The similarity calculation section 103 can obtain the single-beat ventricular waveform from the corrected ventricular waveform in a similar manner.

It should be noted that the similarity calculation section 103 may use a trained model such as a convolutional neural network in order to detect the time points serving as the delimiters of the respective pulsations of the atrium from the corrected atrial waveform. Such a trained model is constructed by machine learning using learning data having, for example, a sample atrial waveform of a sample subject as an explanatory variable and having the time points serving as the delimiters of the respective pulsations of the atrium in the sample atrial waveform as an objective variable. Similarly, the similarity calculation section 103 may use a trained model such as a convolutional neural network in order to detect the time points serving as the delimiters of the respective pulsations of the ventricle from the corrected ventricular waveform. Such a trained model is constructed by machine learning using learning data having, for example, a sample ventricular waveform of a sample subject as an explanatory variable and having the time points serving as the delimiters of the respective pulsations of the ventricle in the sample ventricular waveform as an objective variable.

Next, the similarity calculation section 103 normalizes amplitudes of the obtained plurality of single-beat atrial waveforms and plurality of single-beat ventricular waveforms, and calculates the similarity by comparing the shapes of the plurality of single-beat atrial waveforms after normalization and the shapes of the plurality of single-beat ventricular waveforms after normalization.

For example, if the atrium is in the systole, the ventricle corresponding to the atrium is in the diastole, and if the ventricle is in the systole, the atrium corresponding to the ventricle is in the diastole. Therefore, the similarity calculation section 103 inverts either one of the single-beat atrial waveform and the single-beat ventricular waveform in an increase/decrease direction of the area without changing the shape, and calculates the similarity by comparing the shape of the single-beat atrial waveform and the shape of the single-beat ventricular waveform corresponding to the single-beat atrial waveform.

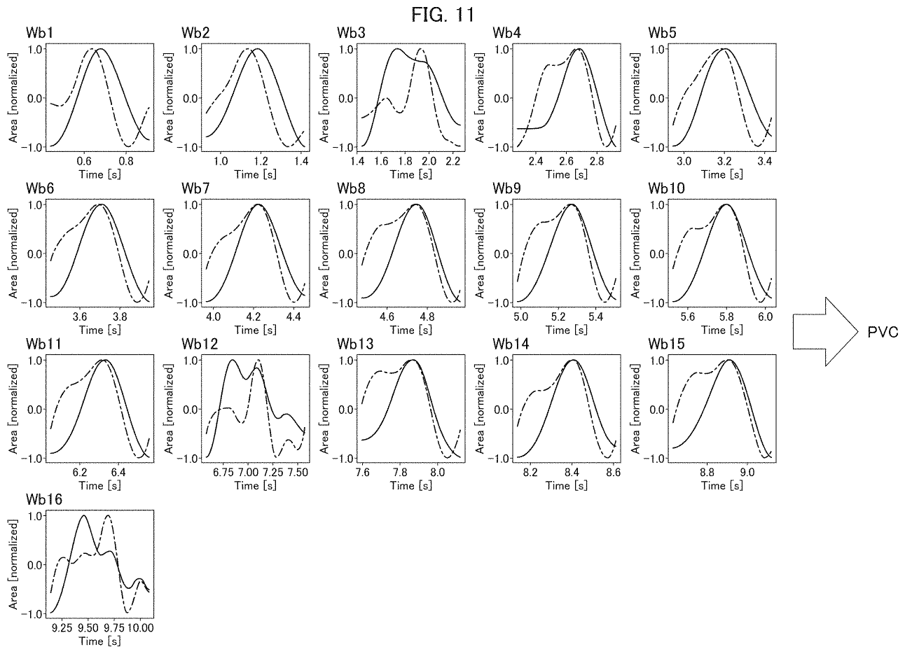

FIGS. 10 and 11 are diagrams each showing an example in which the shapes of respective single-beat atrial waveforms cut out from the atrial waveform and the ventricular waveform of the target signal waveform of different target subjects are compared with the shapes of single-beat ventricular waveforms corresponding to the respective single-beat atrial waveforms. In FIGS. 10 and 11, a dashed-dotted line indicates the single-beat ventricular waveform cut out from the right ventricular waveform, and a solid line indicates the single-beat atrial waveform cut out from the right atrial waveform. For example, in the example shown in FIG. 10, the shapes of the respective single-beat atrial waveforms and the shapes of the single-beat ventricular waveforms corresponding to the respective single-beat atrial waveforms are considerably similar. In such a case, the similarity calculated by the similarity calculation section 103 is high. On the other hand, in the example shown in FIG. 11, the similarity between the shapes of the respective single-beat atrial waveforms and the shapes of the single-beat ventricular waveforms corresponding to the respective single-beat atrial waveforms is lower than that in the example shown in FIG. 10. In such a case, the similarity calculated by the similarity calculation section 103 is low.

The determination section 105a determines the type of a premature contraction suffered by the target subject based on the target signal waveform. Specifically, the determination section 105a determines the type of the premature contraction suffered by the target subject based on the calculated similarity. More specifically, the determination section 105a may determine the type of the premature contraction suffered by the target subject based on a result of comparing the similarity in the period of interest with a predetermined reference value capable of distinguishing between a similarity when PVC is occurring and a similarity when PAC is occurring. That is, the determination section 105a may determine that the target subject is suffering from PVC when the similarity is equal to or less than the predetermined reference value in the period of interest, and may determine that the target subject is suffering from PAC when the similarity is higher than the predetermined reference value in the period of interest. For example, the determination section 105a determines that the type of the premature contraction suffered by the target subject is PAC based on the similarity between the shape of the single-beat atrial waveform and the shape of the single-beat ventricular waveform shown in FIG. 10. On the other hand, the determination section 105a determines that the type of the premature contraction suffered by the target subject is PVC based on the similarity between the shape of the single-beat atrial waveform and the shape of the single-beat ventricular waveform shown in FIG. 11.

### Processing Performed by Arrhythmia Type Determination Device 1a

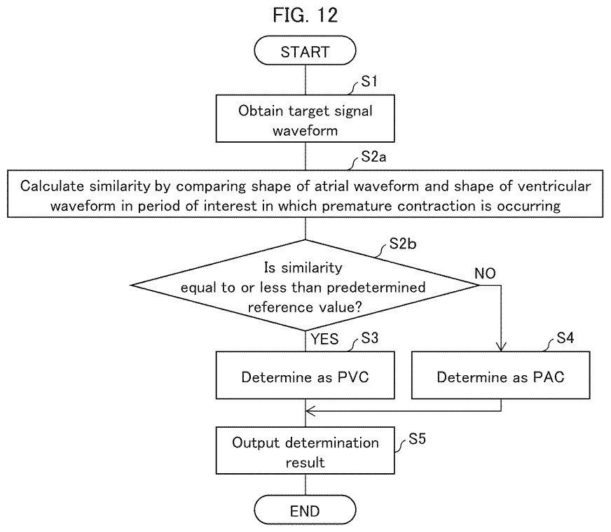

Next, processing (arrhythmia type determination method) performed by the arrhythmia type determination device 1a will be described with reference to FIG. 12. FIG. 12 is a flowchart showing an example of a flow of processing performed by the arrhythmia type determination device 1a.

First, the obtaining section 101 obtains the target signal waveform (step S1: obtaining step). Next, the similarity calculation section 103 calculates the similarity by comparing the shape of the atrial waveform and the shape of the ventricular waveform in the period of interest in which the premature contraction is occurring (step S2a). Then, when the calculated similarity is equal to or less than the predetermined reference value (YES in step S2b), the determination section 105a determines that the premature contraction suffered by the target subject is PVC (step S3: determination step). On the other hand, when the calculated similarity is not equal to or less than the predetermined reference value (NO in step S2b), the determination section 105a determines that the premature contraction suffered by the target subject is PAC (step S4: determination step). The output control section 106 causes an output device (for example, a display device 6) to output a determination result by the determination section 105a (step S5: output step).

With the above configuration, the arrhythmia type determination device 1a can accurately determine the type of the premature contraction suffered by the target subject from an image obtained by image-capturing of the heart of the target subject. For example, the arrhythmia type determination device 1a can estimate whether the type of the premature contraction of a fetus is “PVC” or “PAC” with a high accuracy rate.

### Embodiment 3

Another embodiment of the present invention will be discussed below. For convenience of description, members having the same functions as the members described in the above embodiment are denoted by the same reference numerals, and description thereof will not be repeated.

An arrhythmia type determination device 1b according to the present embodiment is configured to determine, from the target signal waveform, the type of the premature contraction suffered by the target subject based on presence or absence of (1) a deletion of a ventricular contraction corresponding to an atrial contraction and (2) a deletion of an atrial contraction corresponding to a ventricular contraction.

Similarly to the above embodiment, the following description will discuss, as an example, the arrhythmia type determination device 1b having a function of determining the type of the premature contraction suffered by the target subject from the target signal waveform obtained from the premature contraction detection device 4. However, the arrhythmia type determination device 1b may also have the function of the image analysis device 3 and the function of the premature contraction detection device 4. In this case, the arrhythmia type determination device 1b generates the target signal waveform from an input image obtained from the image capturing device 2, and determines the type of the premature contraction suffered by the target subject.

The following description will discuss, as an example, a configuration in which the arrhythmia type determination device 1b obtains a partial waveform (that is, the target signal waveform in the period of interest) cut out by the premature contraction detection device 4, and determines the type of the premature contraction occurring in the heart of the target subject based on the target signal waveform.

### Configuration of Arrhythmia Type Determination Device 1b

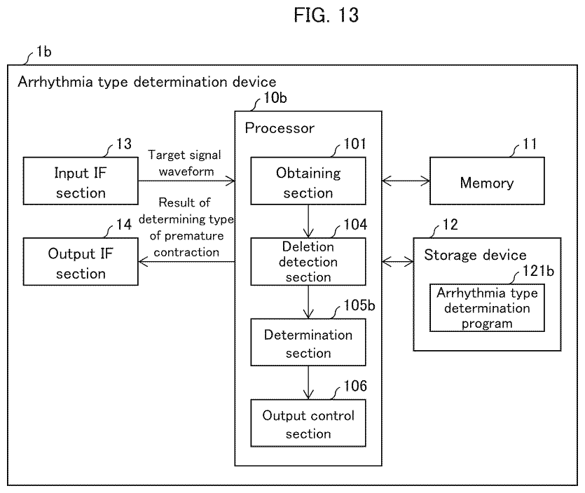

A configuration of the arrhythmia type determination device 1b according to an embodiment of the present invention will be described with reference to FIG. 13. FIG. 13 is a block diagram showing an example of the configuration of the main parts of the arrhythmia type determination device 1b.

As illustrated, the arrhythmia type determination device 1b is, for example, a computer including a processor 10b, the memory 11, and the storage device 12. The arrhythmia type determination device 1b may be a personal computer, a server, or a workstation. The processor 10b functions as each section from the obtaining section 101 to the output control section 106 described later by loading an arrhythmia type determination program 121b stored in the storage device 12 into the memory 11 and executing the arrhythmia type determination program 121b.

The processor 10b can be achieved by a logic circuit (hardware) formed in an integrated circuit (IC chip) or the like, or can be achieved by software. When achieved by software, the processor 10b may be configured by, for example, a CPU, may be configured by a GPU, or may be configured by a combination thereof. In this case, the software is stored in the storage device 12. Then, the processor 10b reads the software into the memory 11 and executes the software.

The processor 10b functions as each of the obtaining section 101 that obtains the target signal waveform from the premature contraction detection device 4, a deletion detection section 104, a determination section 105b, and the output control section 106 by executing the arrhythmia type determination program 121b.

The deletion detection section 104 detects, in the period of interest, a deletion of a ventricular contraction indicated by the ventricular waveform corresponding to an atrial contraction indicated by the atrial waveform, and a deletion of an atrial contraction indicated by the atrial waveform corresponding to a ventricular contraction indicated by the ventricular waveform. Alternatively, the deletion detection section 104 may detect, in the period of interest, a deletion of a ventricular expansion indicated by the ventricular waveform corresponding to an atrial expansion indicated by the atrial waveform, and a deletion of an atrial expansion indicated by the atrial waveform corresponding to a ventricular expansion indicated by the ventricular waveform. Hereinafter, a configuration in which the deletion detection section 104 detects the deletion of the atrial contraction and the ventricular contraction will be described as an example.

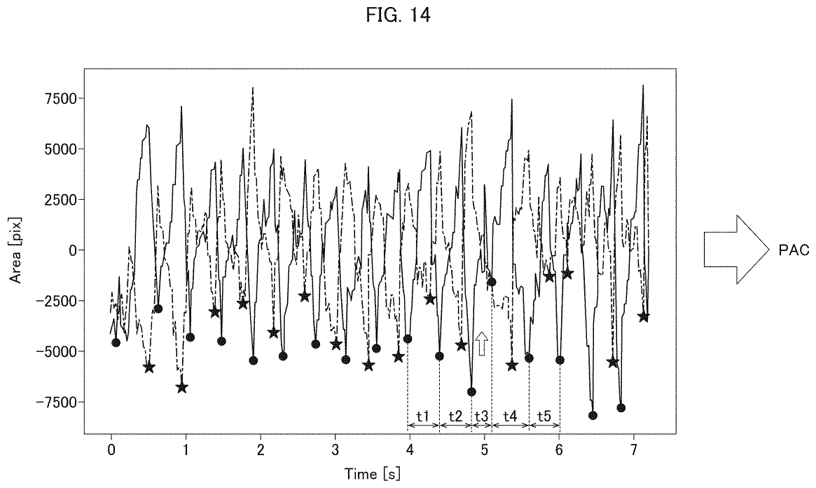

An example of processing performed by the deletion detection section 104 will be described with reference to FIG. 14. FIG. 14 is a diagram showing a state in which the deletion of the ventricular contraction corresponding to the atrial contraction is detected from a set of the atrial waveform and the ventricular waveform. In FIG. 14, a value on a vertical axis indicates a magnitude of an area change at the time of contraction and at the time of expansion of each region of the atrium and the ventricle. In order to detect a deletion to be detected, the deletion detection section 104 first detects, in the target signal waveform, time points V1 at which the atrium turns from the systole to the diastole (local minimum points in the atrial waveform, black circles in FIG. 14) and time points V2 at which the ventricle turns from the systole to the diastole (local minimum points in the ventricular waveform, black stars in FIG. 14).

For example, in FIG. 14, the deletion detection section 104 detects that there is an arrangement of “black circle->black circle->” (white arrow near 5 seconds) in an arrangement of “black circle->black star->black circle->. . . ” over time, and that a black star that should originally exist is deleted. That is, in the example shown in FIG. 14, there exists the deletion of the ventricular contraction indicated by the ventricular waveform corresponding to the atrial contraction indicated by the atrial waveform.

The determination section 105b determines the type of the premature contraction suffered by the target subject based on the target signal waveform. Specifically, the determination section 105b determines the type of the premature contraction suffered by the target subject based on whether the deletion of the ventricular contraction corresponding to the atrial contraction is detected or the deletion of the atrial contraction corresponding to the ventricular contraction is detected in the period of interest. More specifically, the determination section 105b determines that the target subject is suffering from PVC when the deletion of the atrial contraction corresponding to the ventricular contraction is detected in the period of interest. On the other hand, the determination section 105b determines that the target subject is suffering from PAC when the deletion of the ventricular contraction corresponding to the atrial contraction is detected in the period of interest. For example, from the example shown in FIG. 14, the determination section 105b determines that the target subject is suffering from PAC.

It should be noted that the deletion detection section 104 may specify a time of each contraction of the atrium indicated by the atrial waveform and a time of each contraction of the ventricle indicated by the ventricular waveform, based on time information corresponding to each of the time points V1 and the time points V2. In this case, the deletion detection section 104 may be configured to calculate any of the following (i) to (iv) and detect at least one of an abnormality in atrial contraction timing and an abnormality in ventricular contraction timing.
(i) Time from when the atrium contracts until when the atrium contracts next.
(ii) Time from when the ventricle contracts until when the ventricle contracts next.
(iii) Time from when the atrium contracts until when the ventricle contracts next.
(iv) Time from when the ventricle contracts until when the atrium contracts next.

For example, times t1 to t5 shown in FIG. 14 indicate the time from when the atrium contracts until when the atrium contracts next. While the times t1, t2, and t5 are approximately 0.4 seconds, the time t3 is approximately 0.25 seconds and the time t4 is approximately 0.65 seconds. That is, the deletion detection section 104 detects that there is an abnormality in the atrial contraction timing from the example shown in FIG. 14. In this case, the determination section 105b may determine that the target subject is suffering from PAC based on the fact that the abnormality in the atrial contraction timing is detected.

Alternatively, the deletion detection section 104 may be capable of detecting both the deletion of the ventricular and atrial contractions and the abnormality in the atrial and ventricular contraction timings. According to this configuration, the type of the premature contraction suffered by the target subject can be determined with higher accuracy.

Processing Performed by Arrhythmia type determination device 1b)

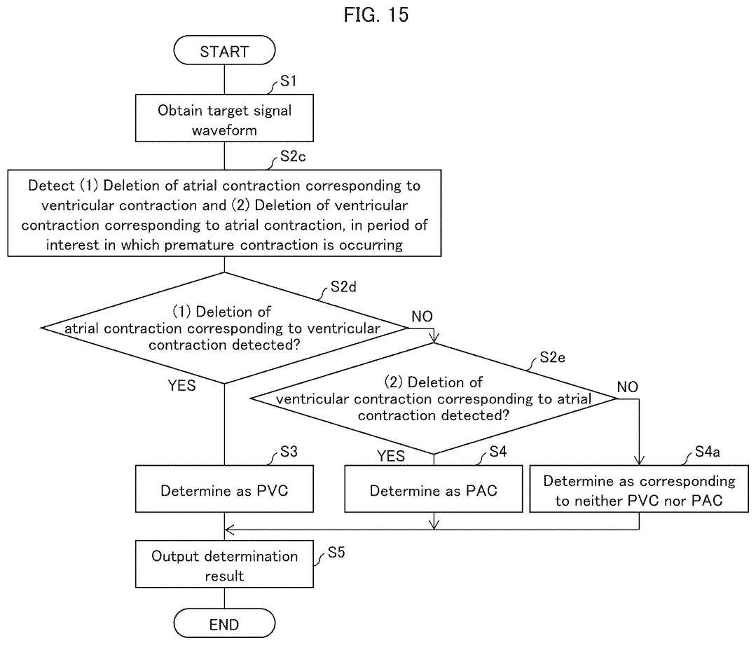

Next, processing (arrhythmia type determination method) performed by the arrhythmia type determination device 1b will be described with reference to FIG. 15. FIG. 15 is a flowchart showing an example of a flow of processing performed by the arrhythmia type determination device 1b.

First, the obtaining section 101 obtains the target signal waveform (step S1: obtaining step).

Next, the deletion detection section 104 detects (1) the deletion of the ventricular contraction corresponding to the atrial contraction and (2) the deletion of the atrial contraction corresponding to the ventricular contraction in the period of interest in which the premature contraction is occurring (step S2c).

When (1) a deletion of the atrial contraction corresponding to a ventricular contraction is detected (YES in step S2d), the determination section 105b determines that the type of the premature contraction suffered by the target subject is PVC (step S3: determination step).

On the other hand, when (1) the deletion of the atrial contraction corresponding to the ventricular contraction is not detected (NO in step S2d) and (2) the deletion of the ventricular contraction corresponding to the atrial contraction is detected (YES in step S2e), the determination section 105b determines that the type of the premature contraction suffered by the target subject is PAC (step S4: determination step). When the result is NO in step S2d and NO in step S2e, the determination section 105b determines that the type of the premature contraction suffered by the target subject is a premature contraction falling under neither PVC nor PAC (step S4a: determination step). It should be noted that in step S4a, the determination section 105b may output to the effect that the type of the premature contraction suffered by the target subject is undeterminable.

Then, the output control section 106 causes the output device (for example, the display device 6) to output the determination result by the determination section 105b (or a message indicating that the determination is impossible) (step S5: output step).

With the above configuration, the arrhythmia type determination device 1b can estimate the type of a tachyarrhythmia suffered by the target subject with higher accuracy from the image obtained by image-capturing of the heart of the target subject. For example, the arrhythmia type determination device 1b can determine whether the type of the premature contraction of the fetus is “PVC” or “PAC” with a high accuracy rate.

### Embodiment 4

Another embodiment of the present invention will be discussed below. For convenience of description, members having the same functions as the members described in the above embodiment are denoted by the same reference numerals, and description thereof will not be repeated.

An arrhythmia type determination device 1c according to the present embodiment includes all of the period detection section 102, the similarity calculation section 103, and the deletion detection section 104 described in Embodiment 3, and is configured to determine the type of the premature contraction suffered by the target subject from the target signal waveform using respective functions.

Similarly to the above embodiment, the following description will discuss, as an example, the arrhythmia type determination device 1c having a function of determining the type of the premature contraction suffered by the target subject from the target signal waveform obtained from the premature contraction detection device 4. However, the arrhythmia type determination device 1c may also have the function of the image analysis device 3 and the function of the premature contraction detection device 4. In this case, the arrhythmia type determination device 1c generates the target signal waveform from the input image obtained from the image capturing device 2, and determines the type of the premature contraction suffered by the target subject.

### Configuration of Arrhythmia Type Determination Device 1c

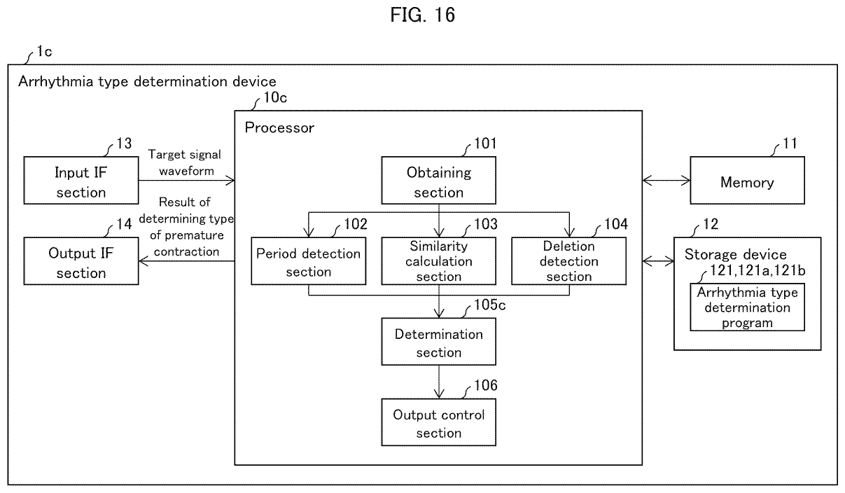

A configuration of the arrhythmia type determination device 1c according to an embodiment of the present invention will be described with reference to FIG. 16. FIG. 16 is a block diagram showing an example of the configuration of the main parts of the arrhythmia type determination device 1c.

As illustrated, the arrhythmia type determination device 1c is, for example, a computer including a processor 10c, the memory 11, and the storage device 12. The arrhythmia type determination device 1c may be a personal computer, a server, or a workstation. The processor 10c functions as each section from the obtaining section 101 to the output control section 106 described later by loading arrhythmia type determination programs 121, 121a, and 121b stored in the storage device 12 into the memory 11 and executing the arrhythmia type determination programs 121, 121a, and 121b.

The processor 10c can be achieved by a logic circuit (hardware) formed in an integrated circuit (IC chip) or the like, or can be achieved by software. When achieved by software, the processor 10c may be configured by, for example, a CPU, may be configured by a GPU, or may be configured by a combination thereof. In this case, the software is stored in the storage device 12. Then, the processor 10c reads the software into the memory 11 and executes the software.

The processor 10c functions as each of the obtaining section 101 that obtains the target signal waveform from the premature contraction detection device 4, the period detection section 102, the similarity calculation section 103, the deletion detection section 104, a determination section 105c, and the output control section 106 by executing the arrhythmia type determination programs 121, 121a, and 121b.

According to this configuration, the determination section 105c may determine the type of the premature contraction suffered by the target subject by three types of processing described in Embodiments 1 to 3, and output three determination results.

Alternatively, the determination section 105c may be configured to output one determination result regarding the type of the premature contraction suffered by the target subject based on a degree of coincidence of the three determination results. This makes it possible to determine the type of the premature contraction suffered by the target subject with higher accuracy. For example, the determination section 105c may be configured to output one determination result regarding the type of the premature contraction suffered by the target subject by a majority vote of the three determination results. That is, when two of the three determination results determine PAC and one determines PVC, the determination section 105c may output one determination result that the type of the premature contraction suffered by the target subject is PAC. In this case, it is preferable that the determination section 105c outputs, together with the determination result, information regarding a fact that there was a possibility that the type of the premature contraction suffered by the target subject was determined to be PVC, and a basis for the determination. This makes it possible to allow a medical worker or the like to confirm correctness of the type of the premature contraction suffered by the target subject determined by the arrhythmia type determination device 1c.

It should be noted that in the present embodiment, the arrhythmia type determination device 1c including all three of the period detection section 102, the similarity calculation section 103, and the deletion detection section 104 has been described. However, the configuration of the arrhythmia type determination device 1c is not limited thereto. For example, the arrhythmia type determination device 1c may include only the period detection section 102 and the similarity calculation section 103, may include only the period detection section 102 and the deletion detection section 104, or may include only the similarity calculation section 103 and the deletion detection section 104.

### Software Implementation Example

The functions of the arrhythmia type determination devices 1, 1a, 1b, and 1c (hereinafter, referred to as a “device”) can be realized by a program for causing a computer to function as the device, the program causing the computer to function as the control blocks (particularly, the sections included in the processors 10, 10a, 10b, and 10c) of the device.

In this case, the device includes a computer that has at least one control device (for example, a processor) and at least one memory device (for example, a memory) as hardware for executing the program. By the control device executing the program with use of the storage device, the functions described in the foregoing embodiments are realized.

The program can be stored in one or more non-transitory computer-readable storage media. The storage medium can be provided in the device, or the storage medium does not need to be provided in the device. In the latter case, the program can be supplied to or made available to the device via any wired or wireless transmission medium.

Alternatively, a part or all of the functions of the control blocks can be realized by a logic circuit. For example, the present invention encompasses, in its scope, an integrated circuit in which a logic circuit that functions as each of the control blocks is formed. In addition, the function of each of the control blocks can be realized by, for example, a quantum computer.

Each of the processes described in the foregoing embodiments may be carried out by artificial intelligence (AI). In this case, the AI may be operated by the control device or may be operated by another device (for example, an edge computer and a cloud server).

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

An arrhythmia type determination device according to Aspect 1 of the present invention includes: an obtaining section that obtains a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and a determination section that determines the type of a premature contraction suffered by the target subject, based on the target signal waveform in a period of interest in which the premature contraction is occurring in the heart of the target subject.

The arrhythmia type determination device according to Aspect 2 of the present invention, in Aspect 1, may be configured such that: the target signal waveform includes an atrial waveform indicating the time-series change in the area of the region of the left atrium or the right atrium and a ventricular waveform indicating the time-series change in the area of the region of the left ventricle or the right ventricle; the arrhythmia type determination device further includes a period detection section that detects, in the period of interest, an increasing period in which both the area indicated by the atrial waveform and the area indicated by the ventricular waveform increase and a decreasing period in which both the area indicated by the atrial waveform and the area indicated by the ventricular waveform decrease; and the determination section determines the type of the premature contraction suffered by the target subject based on a status of detecting the increasing period and the decreasing period in the period of interest.

The arrhythmia type determination device according to Aspect 3 of the present invention, in Aspect 2, may be configured such that the period detection section calculates a corrected atrial waveform and a corrected ventricular waveform which are obtained by removing noise from the atrial waveform and the ventricular waveform, respectively, detects, as the increasing period in the period of interest, a period in which both an area indicated by the corrected atrial waveform and an area indicated by the corrected ventricular waveform increase for a predetermined time or more, and detects, as the decreasing period in the period of interest, a period in which both the area indicated by the corrected atrial waveform and the area indicated by the corrected ventricular waveform decrease for a predetermined time or more.

The arrhythmia type determination device according to Aspect 4 of the present invention, in Aspect 2 or 3, may be configured such that the determination section determines that the target subject is suffering from a premature ventricular contraction when either the increasing period or the decreasing period is detected in the period of interest, and determines that the target subject is suffering from a premature atrial contraction when neither the increasing period nor the decreasing period is detected in the period of interest.

The arrhythmia type determination device according to Aspect 5 of the present invention, in any one of Aspects 1 through 4, may be configured such that: the target signal waveform includes an atrial waveform indicating the time-series change in the area of the region of the left atrium or the right atrium and a ventricular waveform indicating the time-series change in the area of the region of the left ventricle or the right ventricle; the arrhythmia type determination device further includes a similarity calculation section that calculates a similarity by comparing a shape of the atrial waveform and a shape of the ventricular waveform in the period of interest; and the determination section determines the type of the premature contraction suffered by the target subject based on the similarity.

The arrhythmia type determination device according to Aspect 6 of the present invention, in Aspect 5, may be configured such that the similarity calculation section calculates a corrected atrial waveform and a corrected ventricular waveform which are obtained by removing noise from the atrial waveform and the ventricular waveform, respectively, obtains a plurality of single-beat atrial waveforms corresponding to respective single heartbeats of the heart of the target subject from the corrected atrial waveform in the period of interest and obtains a plurality of single-beat ventricular waveforms corresponding to respective single heartbeats of the heart of the target subject from the corrected ventricular waveform in the period of interest, normalizes amplitudes of the obtained plurality of single-beat atrial waveforms and plurality of single-beat ventricular waveforms, and calculates the similarity by comparing shapes of the plurality of single-beat atrial waveforms and shapes of the plurality of single-beat ventricular waveforms which have been normalized.

The arrhythmia type determination device according to Aspect 7 of the present invention, in Aspect 5 or 6, may be configured such that the determination section determines that the target subject is suffering from a premature ventricular contraction when the similarity is equal to or less than a predetermined reference value in the period of interest, and determines that the target subject is suffering from a premature atrial contraction when the similarity is higher than the predetermined reference value in the period of interest.

The arrhythmia type determination device according to Aspect 8 of the present invention, in any one of Aspects 1 through 7, may be configured such that: the target signal waveform includes an atrial waveform indicating the time-series change in the area of the region of the left atrium or the right atrium and a ventricular waveform indicating the time-series change in the area of the region of the left ventricle or the right ventricle; the arrhythmia type determination device further includes a deletion detection section that detects, in the period of interest, a deletion of a ventricular contraction indicated by the ventricular waveform corresponding to an atrial contraction indicated by the atrial waveform and a deletion of an atrial contraction indicated by the atrial waveform corresponding to a ventricular contraction indicated by the ventricular waveform; and the determination section determines the type of the premature contraction suffered by the target subject based on, in the period of interest, whether the deletion of the ventricular contraction corresponding to the atrial contraction is detected or the deletion of the atrial contraction corresponding to the ventricular contraction is detected.

The arrhythmia type determination device according to Aspect 9 of the present invention, in Aspect 8, may be configured such that the determination section determines that the target subject is suffering from a premature ventricular contraction when the deletion of the atrial contraction corresponding to the ventricular contraction is detected in the period of interest, and determines that the target subject is suffering from a premature atrial contraction when the deletion of the ventricular contraction corresponding to the atrial contraction is detected in the period of interest.

An arrhythmia type determination method according to Aspect 10 of the present invention is executed by one or more information processing devices, the method including: an obtaining step of obtaining a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and a determination step of determining the type of a premature contraction suffered by the target subject, based on the target signal waveform in a period of interest in which the premature contraction is occurring in the heart of the target subject.

A non-transitory computer-readable recording medium according to Aspect 11 of the present invention records an arrhythmia type determination program for causing a computer to function as the arrhythmia type determination device according to any one of Aspects 1 through 9, the arrhythmia type determination program causing the computer to function as the obtaining section and the determination section.

### REFERENCE SIGNS LIST

1, 1a, 1b, 1c Arrhythmia type determination device
10, 10a, 10b, 10c Processor
101 Obtaining section
102 Period detection section
103 Similarity calculation section
104 Deletion detection section
105, 105a, 105b, 105c Determination section
121, 121a, 121b Arrhythmia type determination Program

1. An arrhythmia type determination device comprising:
an obtaining section that obtains a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and
a determination section that determines the type of a premature contraction suffered by the target subject, based on the target signal waveform in a period of interest in which the premature contraction is occurring in the heart of the target subject.

2. The arrhythmia type determination device according to claim 1, wherein:
the target signal waveform includes an atrial waveform indicating the time-series change in the area of the region of the left atrium or the right atrium and a ventricular waveform indicating the time-series change in the area of the region of the left ventricle or the right ventricle;
the arrhythmia type determination device further comprises a period detection section that detects, in the period of interest, an increasing period in which both the area indicated by the atrial waveform and the area indicated by the ventricular waveform increase and a decreasing period in which both the area indicated by the atrial waveform and the area indicated by the ventricular waveform decrease; and
the determination section
determines the type of the premature contraction suffered by the target subject based on a status of detecting the increasing period and the decreasing period in the period of interest.

3. The arrhythmia type determination device according to claim 2, wherein
the period detection section
calculates a corrected atrial waveform and a corrected ventricular waveform which are obtained by removing noise from the atrial waveform and the ventricular waveform, respectively,
detects, as the increasing period in the period of interest, a period in which both an area indicated by the corrected atrial waveform and an area indicated by the corrected ventricular waveform increase for a predetermined time or more, and
detects, as the decreasing period in the period of interest, a period in which both the area indicated by the corrected atrial waveform and the area indicated by the corrected ventricular waveform decrease for a predetermined time or more.

4. The arrhythmia type determination device according to claim 2, wherein
the determination section
determines that the target subject is suffering from a premature ventricular contraction when either the increasing period or the decreasing period is detected in the period of interest, and
determines that the target subject is suffering from a premature atrial contraction when neither the increasing period nor the decreasing period is detected in the period of interest.

5. The arrhythmia type determination device according to claim 1, wherein:
the target signal waveform includes an atrial waveform indicating the time-series change in the area of the region of the left atrium or the right atrium and a ventricular waveform indicating the time-series change in the area of the region of the left ventricle or the right ventricle;
the arrhythmia type determination device further comprises a similarity calculation section that calculates a similarity by comparing a shape of the atrial waveform and a shape of the ventricular waveform in the period of interest; and
the determination section
determines the type of the premature contraction suffered by the target subject based on the similarity.

6. The arrhythmia type determination device according to claim 5, wherein
the similarity calculation section
calculates a corrected atrial waveform and a corrected ventricular waveform which are obtained by removing noise from the atrial waveform and the ventricular waveform, respectively,
obtains a plurality of single-beat atrial waveforms corresponding to respective single heartbeats of the heart of the target subject from the corrected atrial waveform in the period of interest and
obtains a plurality of single-beat ventricular waveforms corresponding to respective single heartbeats of the heart of the target subject from the corrected ventricular waveform in the period of interest,
normalizes amplitudes of the obtained plurality of single-beat atrial waveforms and plurality of single-beat ventricular waveforms, and
calculates the similarity by comparing shapes of the plurality of single-beat atrial waveforms and shapes of the plurality of single-beat ventricular waveforms which have been normalized.

7. The arrhythmia type determination device according to claim 5, wherein
the determination section
determines that the target subject is suffering from a premature ventricular contraction when the similarity is equal to or less than a predetermined reference value in the period of interest, and
determines that the target subject is suffering from a premature atrial contraction when the similarity is higher than the predetermined reference value in the period of interest.

8. The arrhythmia type determination device according to claim 1, wherein:
the target signal waveform includes an atrial waveform indicating the time-series change in the area of the region of the left atrium or the right atrium and a ventricular waveform indicating the time-series change in the area of the region of the left ventricle or the right ventricle;
the arrhythmia type determination device further comprises a deletion detection section that detects, in the period of interest, a deletion of a ventricular contraction indicated by the ventricular waveform corresponding to an atrial contraction indicated by the atrial waveform and a deletion of an atrial contraction indicated by the atrial waveform corresponding to a ventricular contraction indicated by the ventricular waveform; and
the determination section
determines the type of the premature contraction suffered by the target subject based on, in the period of interest, whether the deletion of the ventricular contraction corresponding to the atrial contraction is detected or the deletion of the atrial contraction corresponding to the ventricular contraction is detected.

9. The arrhythmia type determination device according to claim 8, wherein
the determination section
determines that the target subject is suffering from a premature ventricular contraction when the deletion of the atrial contraction corresponding to the ventricular contraction is detected in the period of interest, and
determines that the target subject is suffering from a premature atrial contraction when the deletion of the ventricular contraction corresponding to the atrial contraction is detected in the period of interest.

10. An arrhythmia type determination method executed by one or more information processing devices,
the method comprising:
an obtaining step of obtaining a target signal waveform including at least one of a set of a signal waveform indicating a time-series change in an area of a region of a left atrium and a signal waveform indicating a time-series change in an area of a region of a left ventricle and a set of a signal waveform indicating a time-series change in an area of a region of a right atrium and a signal waveform indicating a time-series change in an area of a region of a right ventricle, which are generated by analyzing an image of a heart of a target subject; and
a determination step of determining the type of a premature contraction suffered by the target subject, based on the target signal waveform in a period of interest in which the premature contraction is occurring in the heart of the target subject.

11. A non-transitory computer-readable recording medium recording an arrhythmia type determination program for causing a computer to function as the arrhythmia type determination device according to claim 1, the arrhythmia type determination program causing the computer to function as the obtaining section and the determination section.
